(19) United States
(12) Patent Application Publication
(10) Pub. No.: US 2020/0360171 A1
(43) Pub. Date: Nov. 19, 2020

(54) UNIVERSAL SIZE VACUUM SEALED WATERPROOF CAST AND BANDAGE COVER

(71) Applicant: HMP Enterprises, Inc., Manchester, GA (US)

(72) Inventor: Roy Archambault, Wilmington, NC (US)

(21) Appl. No.: 16/411,001

(22) Filed: May 13, 2019

(51) Int. Cl. A61F 5/058

(52) U.S. Cl. CPC A61F 5/05816; A61F 5/05858; A61F 5/0585

(57) ABSTRACT

A method and apparatus includes an elastic cover configured to be positioned around a bandaged leg. A pump may be positioned within the elastic cover and configured to evacuate air from an interior space defined by the elastic cover when the elastic cover is positioned around the leg.

### BACKGROUND

The present invention relates generally to medical devices, and more particularly, to methods to apply a cast or bandage to an injured limb or extremity.

Hot and cold packs are used to relieve pain and encourage healing. Conventional packs may slip off of a targeted area of a patient, or may interfere with compression therapy.

### SUMMARY

According to a particular embodiment, an apparatus includes an elastic cover configured to be positioned around a body part. The apparatus further includes a pump positioned within the cover and configured to evacuate or introduce air into the cover.

### BRIEF DESCRIPTION OF THE DRAWINGS

FIG. 1 is a side, cut-away view of an embodiment of a universal bandage cover configured to fit around and seal a cast or other cover worn on a hand.

FIG. 2 shows a side, cut-away view of an embodiment of a universal bandage cover configured to fit around and seal a cast or bandage worn on a foot.

### DETAILED DESCRIPTION

An embodiment of a system may include a cover that accommodates dozens of conventional sizes for various sized users. The universal cover may solve the problem of manufacturing, supply chain and retailers having to deal with dozens of items to manage. The system may also make the purchasing process easier for end-users only having to select one size versus having to measure and choose from many sizes.

The sizes of embodiments of the cover may be determined based on empirical data regarding cast, bandage, and limb sizes. This data may be used to determine a universal arm size and a universal leg size using a computing algorithm. The universal arm opening part of an embodiment of the cover may range from 6 to 8.5 inches for the arm size with a total length of 24 inches. The universal leg opening part of the cover may range from 9.25 to 11 inches for the leg size with a total length of 28 inches. In order to fit the variety range of people, the cover may be made of a thinner (e.g., ranging 0.2 mm to 0.55 mm in thickness) and more elastic material to enable a stretch up to 300%.

The cover system may include a pump and valve welded into a cover as one unit. The pump of an embodiment may be a cigar shaped and attached to the inside of the cover. This configuration contrasts with an embodiment where the pump is only temporarily attached, and may avoid occurrences where the pump could fall off and be lost. The leg cover system may have a special additional layer of ground up rubber-like material attached on the bottom to provide a rugged surface to prevent slipping and puncturing while in use.

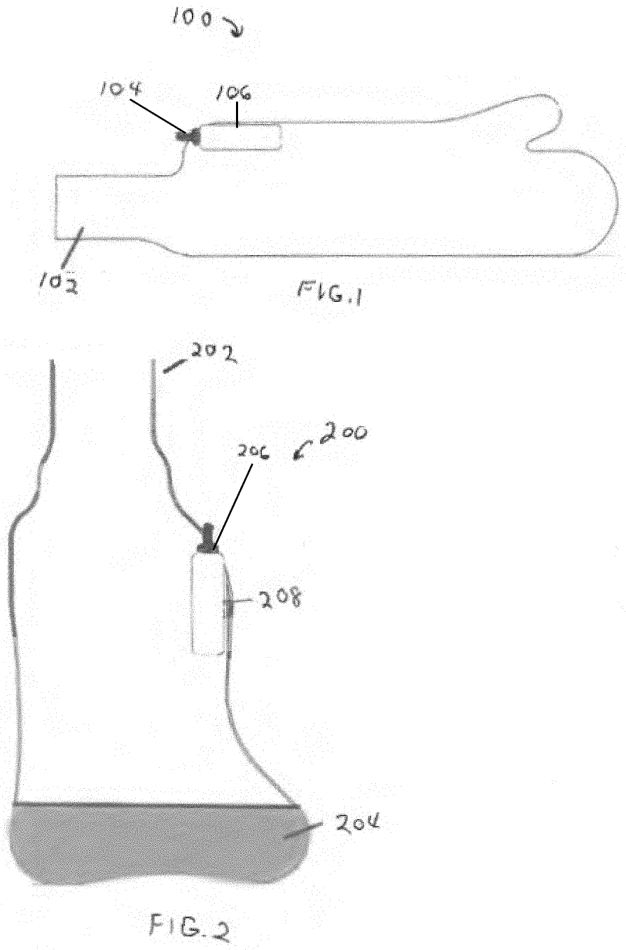

Now turning to the Drawings, FIG. 1 is a side, cut-away view of an embodiment of a universal bandage cover system 100 configured to fit around and seal a cast or other cover worn on a hand. The system 100 includes a cover 102 made from a thin, elastic material. The system 100 also includes a pump 106 and a one-way check valve 104. The pump 106 may be used to evacuate air and create a waterproof seal once the cover 102 is positioned around the arm.

FIG. 2 shows a side, cut-away view of an embodiment of a universal bandage cover system configured to fit around and seal a cast or bandage worn on a foot. The system 200 includes a cover 102 made from a thin, elastic material. The system 200 may include a textured, non-skid coating or other material 204. The system 200 also includes a pump 208 and a one-way check valve 206. The pump 208 may be used to evacuate air and create a waterproof seal once the cover 202 is positioned around the foot.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the preceding, reference is made to embodiments presented in this disclosure. However, the scope of the present disclosure is not limited to specific described embodiments. Instead, any combination of the features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the aspects, features, embodiments and advantages described above are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

While the foregoing is directed to embodiments of the present invention, other and further embodiments of the invention may be devised without departing from the basic scope thereof, and the scope thereof is determined by the claims that follow.

1. An apparatus comprising:
an elastic cover configured to be positioned around a leg, the elastic cover defining an open top end and a closed bottom end;
a pump positioned within the elastic cover and configured to evacuate air from an interior space defined by the elastic cover when the elastic cover is positioned around the leg; and
a non-skid layer attached to the bottom end, the non-skid layer configured to prevent slipping and puncturing when the elastic cover is in use.

2. The apparatus of claim 1, wherein the non-skid layer comprises ground-up rubber-like material.

3. The apparatus of claim 1, wherein the pump is cigar-shaped.

4. The apparatus of claim 1, wherein the pump is welded to the inside of the elastic cover.

5. The apparatus of claim 1, further comprising a one-way check valve positioned within the elastic cover.

6. The apparatus of claim 1, wherein the open top end defines an opening ranging from and including 9.25 inches to and including 11 inches.

7. The apparatus of claim 1, wherein the elastic cover has a thickness ranging from and including 0.2 mm to and including 0.55 mm.

8. The apparatus of claim 1, wherein the elastic cover can be stretched up to 300% from its original size.

9. A method of covering a leg, comprising the steps of:
positioning an elastic cover around a leg, the elastic cover defining an open top end and a closed bottom end, the elastic cover further defining an interior space when positioned around the leg;
evacuating air from the interior space by using a pump and a check valve positioned within the elastic cover; and
preventing skidding of the leg with a non-skid layer attached to the bottom end of the elastic cover.

10. The method of claim 9, wherein the non-skid layer comprises ground-up rubber-like material.

11. The method of claim 9, wherein the pump is cigar-shaped.

12. The method of claim 9, wherein the pump is welded to the inside of the elastic cover.

13. The method of claim 9, wherein the open top end defines an opening ranging from and including 9.25 inches to and including 11 inches.

14. The method of claim 9, wherein the elastic cover has a thickness ranging from and including 0.2 mm to and including 0.55 mm.

15. The method of claim 9, wherein the step of positioning comprises stretching the elastic cover up to 300% from its original size.
